(19) United States
(12) Patent Application Publication
(10) Pub. No.: US 2023/0093159 A1
(43) Pub. Date: Mar. 23, 2023

(54) D-Leefer, Bucked Cannabis Flower and Biomass Refinement Apparatus and Kief Extractor System

(71) Applicant: Thomas Bruggemann, Chatsworth, CA (US)

(72) Inventor: Thomas Bruggemann, Chatsworth, CA (US)

(21) Appl. No.: 17/448,207

(22) Filed: Sep. 21, 2021

(51) Int. Cl. B07B 4/08; B07B 1/20; A01H 6/28

(52) U.S. Cl. CPC B07B 4/08; B07B 1/20; A01H 6/28

(57) ABSTRACT

The D-Leefer, Bucked Cannabis Flower and Biomass Refinement Apparatus and Kief Extractor System is a device for separating dried leaves from female flowers or for separating and refining a plurality of leaves and a plurality of kief from a biomass comprising a hopper having a tray in the form a U-shaped channel having a plurality of openings positioned along its bottom, an auger positioned within said tray having a auger drive motor to spin said auger about its length, a mesh positioned along the bottom half of said auger, a plurality of collection hoods positioned along the bottom side of said U-shaped tray under said mesh each having a vacuum connection, and a plurality of vacuum devices each having a collection container and a hose attached to said vacuum connections.

### TECHNICAL FIELD OF THE INVENTION

This invention relates generally to a Biomass Refinement Apparatus in the form of a hopper feed rotating auger and mesh combination with vacuum extraction enabling collection of fine or powdery plant particles to separate various classifications of biomass material such as stalks, stems, leaves, flowers, and other plant based elements as defined in the industry of said plant.

This invention relates generally to a Cannabis Biomass Refinement Apparatus in the form of a hopper feed rotating auger and mesh combination with vacuum extraction enabling collection of fine or powdery plant particles to separate various classifications of biomass material such as stalks, stems, leaves, flowers, kief, and other plant based elements as defined in the cannabis industry.

This invention relates more specifically to a Cannabis Biomass Refinement Apparatus and Kief Extractor System in the form of a hopper feed rotating auger and mesh combination for dry sluicing with vacuum extraction enabling collection of fine plant particles such as kief to separate various classifications of biomass material such as stalks, stems, leaves, flowers, and other plant based elements as defined in the industry of said plant, such as kief.

This invention relates more specifically to a Bucked Cannabis Flower Refinement Apparatus in the form of a hopper feed rotating auger and mesh combination with vacuum extraction enabling removal and collection of cannabis water (fan) leaves wherein said water leaves are removed from cannabis flowers bucked off the plant loaded while the bucked cannabis flowers are moved out of the hopper by the rotating auger and progress through the rotating auger exiting an open end of said rotating auger without damaging the cannabis flowers.

### BACKGROUND

Generally today the water (fan) leaves are removed from cannabis flowers bucked-off the cannabis plant by hand to help ensure that the cannabis flowers are not damaged.

There are devices that process biomasses at various stages. There are devices that help with handling various components of biomasses in bulk.

There are no machines, apparatuses, or devices that can separate and sort the valuable fine or powdery plant particles from cannabis biomass. At the time of this writing, the valuable fine or powdery plant particles from cannabis biomass are the dried leaves, and kief.

There are no machines, apparatuses, or devices that can remove and collect the water (fan) leaves from cannabis flowers bucked-off the cannabis plant in bulk without damaging the cannabis flowers.

In light of the foregoing prior art, there is a need for a device that can separate, sort, and collect the valuable fine or powdery plant particles from cannabis biomass to better separate the valuable fine or powdery plant particles from cannabis biomass from the cannabis biomass.

In light of the foregoing prior art, there is a need for a device that can remove and collect the water (fan) leaves from cannabis flowers bucked-off the cannabis plant in bulk without damaging the cannabis flowers.

In light of the foregoing prior art, there is a need for a device that can continuously separate, sort, and collect the valuable fine or powdery plant particles from cannabis biomass to better separate the valuable fine or powdery plant particles from cannabis biomass from the cannabis biomass.

In light of the foregoing prior art, there is a need for a device that can continuously remove and collect the water (fan) leaves from cannabis flowers bucked-off the cannabis plant in bulk without damaging the cannabis flowers.

### BRIEF SUMMARY OF THE INVENTION

According to a first aspect of the invention there is a Bucked Cannabis Flower and Biomass Refinement Apparatus comprising a deleafing device for separating dried leaves from female flowers comprising a hopper having a tray in the form a U-shaped channel having a plurality of openings positioned along a bottom with a lid covering a top side of said U-shaped channel connectively attached thereto, an auger positioned within said tray having a auger drive motor operationally attached to spin said auger about a length, a first mesh positioned around up to all of a bottom half of said length of said auger, a plurality of collection hoods positioned along a bottom side of said tray under said first mesh each having a vacuum connection, and a plurality of vacuum devices each having a collection container and a hose operationally attached to said vacuum connections.

According to a second aspect of the invention there is a Bucked Cannabis Flower and Biomass Refinement Apparatus as in the first aspect wherein said first mesh is position in a tube shape around said length of said auger and said first mesh further comprises a mesh drive motor operationally attached to said first mesh to spin said first mesh thereby enabling an increased speed of operation of said deleafing device.

According to a third aspect of the invention there is a Bucked Cannabis Flower and Biomass Refinement Apparatus as in the first aspect further comprising a second mesh having a smaller opening than said first mesh positioned under or replacing a portion of said first mesh enabling a separate vacuum device collection.

According to a fourth aspect of the invention there is a method of using a deleafing device to separate a plurality of dried leaves from a female cannabis flower of a cannabis plant comprising adding a plurality of female cannabis flowers striped off of a branch of a cannabis plant into a hopper, rotating an auger to propel said female cannabis flowers through a U-shaped channel causing said female cannabis flowers to pass over and rub against a first mesh positioned over a vacuum connection causing the separation of said dried leaves from said female cannabis flowers, and operating a plurality of vacuum devices during said rotating to collect said dried leaves.

According to a fifth aspect of the invention there is a biomass separation and refinement deleafing device for separating and refining a plurality of leaves and a plurality of kief from a biomass, consisting of all parts of a cannabis plant, comprising a hopper having a tray in the form a U-shaped channel having a plurality of openings positioned along a bottom with a lid covering a top side of said U-shaped channel connectively attached thereto, an auger positioned within said tray having a auger drive motor operationally attached to spin said auger about a length, a first mesh positioned around up to all of a bottom half of said length of said auger, a plurality of collection hoods positioned along a bottom side of said tray under said first mesh each having a vacuum connection, and a first plurality of vacuum devices and a second plurality of vacuum devices each having a collection container and a hose operationally attached to said vacuum connections.

According to a sixth aspect of the invention there is a deleafing device as in the fifth aspect wherein said first mesh is position in a tube shape around said length of said auger and said first mesh further comprises a mesh drive motor operationally attached to said first mesh to spin said first mesh thereby enabling an increased speed of operation of said deleafing device.

According to a seventh aspect of the invention there is a deleafing device as in the fifth aspect further comprising a second mesh having a smaller opening than said first mesh positioned under or replacing a portion of said first mesh enabling a separate vacuum device collection.

According to an eighth aspect of the invention there is a method of using a cannabis flower and biomass separation and refinement device to separate said plurality of leaves and said plurality of kief from a biomass comprising adding a biomass of a cannabis plant into a hopper, rotating an auger to propel said biomass through said U-shaped channel causing said biomass to pass over and rub against a first mesh positioned over a vacuum connection causing the separation of said plurality of leaves and said plurality of kief from said biomass, and operating a plurality of vacuum devices during said rotating to collect said plurality of leaves and said plurality of kief in separate vacuum devices.

According to a ninth aspect of the invention there is a method as in the eighth aspect wherein said rotating an auger propels said biomass through a U-shaped channel causing said biomass to pass over and rub against a first mesh and a second mesh positioned over a vacuum connection causing the separation of said dried leaves and kief from said biomass, and operating a plurality of vacuum devices during said rotating to collect said dried leaves and said kief in separate vacuum devices.

An advantage of the Bucked Cannabis Flower and Biomass Refinement Apparatus and Kief Extractor System is to enable continuous operation of the automated removal of water (fan) leaves from cannabis flowers without damaging the cannabis flowers.

An advantage of the Bucked Cannabis Flower and Biomass Refinement Apparatus and Kief Extractor System is to enable continuous operation of the automated removal of dried leaves and kief from a cannabis biomass.

The invention will now be described, by way of example only, with reference to the accompanying drawings in which:

### BRIEF DESCRIPTION OF THE DRAWINGS

FIG. 1 is a perspective view of the Biomass Refinement System and Kief Extractor according to the invention;

FIG. 2 is a exploded perspective view of the elements of the Biomass Refinement System and Kief Extractor according to the invention;

FIG. 3 an overhead view of the Biomass Refinement System and Kief Extractor according to the invention; and

FIG. 4 a side view of the Biomass Refinement System and Kief Extractor according to the invention.

### DETAILED DESCRIPTION

The detailed embodiments of the present invention are disclosed herein. The disclosed embodiments are merely exemplary of the invention, which may be embodied in various forms. The details disclosed herein are not to be interpreted as limiting, but merely as the basis for the claims and as a basis for teaching one skilled in the art how to make and use the invention.

References in the specification to “one embodiment,” “an embodiment,” “an example embodiment,” etcetera, indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to effect such feature, structure, or characteristic in connection with other embodiments whether or not explicitly described.

Furthermore, it should be understood that spatial descriptions (e.g., “above,” “below,” “up,” “left,” “right,” “down,” “top,” “bottom,” “vertical,” “horizontal,” etc.) used herein are for purposes of illustration only, and that practical implementations of the structures described herein can be spatially arranged in any orientation or manner.

Throughout this specification, the word “comprise”, or variations thereof such as “comprises” or “comprising”, will be understood to imply the inclusion of a stated element, integer or step, or group of elements integers or steps, but not the exclusion of any other element, integer or step, or group of elements, integers or steps.

Throughout this specification and claims, the phrase “dried leaves” includes any combination of cannabis plant material components including, but not limited to the following: water leaves, fan leaves, and sugar leaves. Note: sugar leaves may still be attached to flowers.

Throughout this specification and claims, the phrase “valuable trim” includes any combination of commercially viable components of cannabis plant material.

Throughout this specification and claims, the phrase “continuous feed” will be understood to imply continuously adding and/or conveying more material to be processed through a machine into its hopper or other raw material input retaining apparatus to ensure that the machine doing the processing can continue to operate without interruption.

Throughout this specification and claims, the phrase “bucked cannabis flower”, or variations thereof such as “bucked cannabis flowers”, will be understood to imply the use of the bucking process for cannabis flowers wherein is the bucking process means stripping off and isolating the cannabis flowers and water leaves from the branches of the cannabis plant.

Index of Labelled Features in Figures. Features are listed in numeric order by Figure in numeric order.

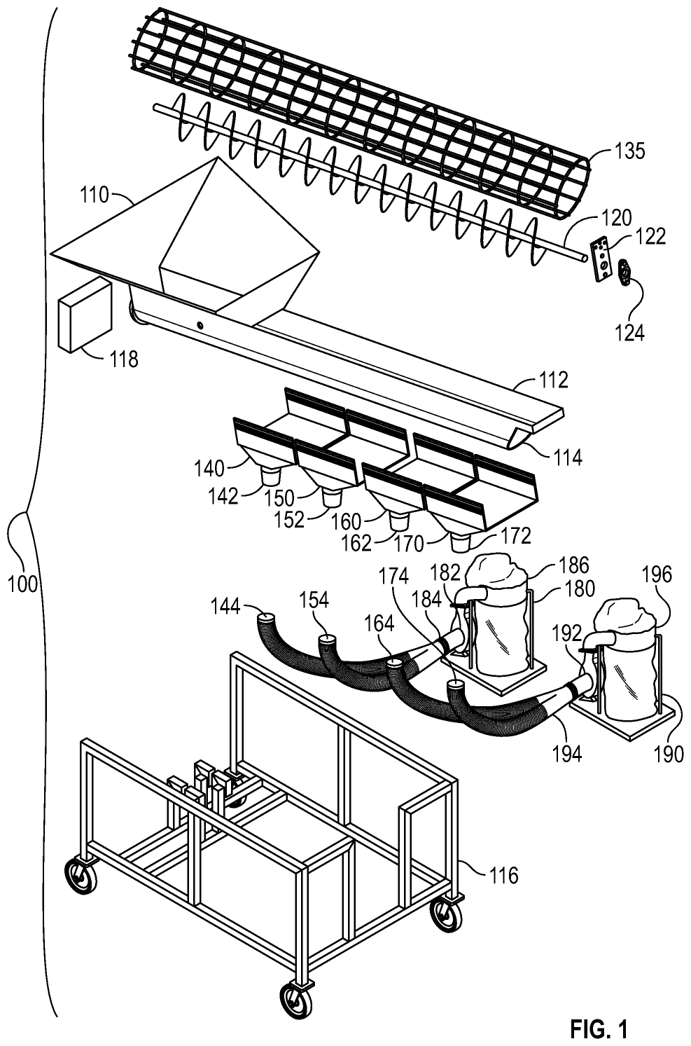

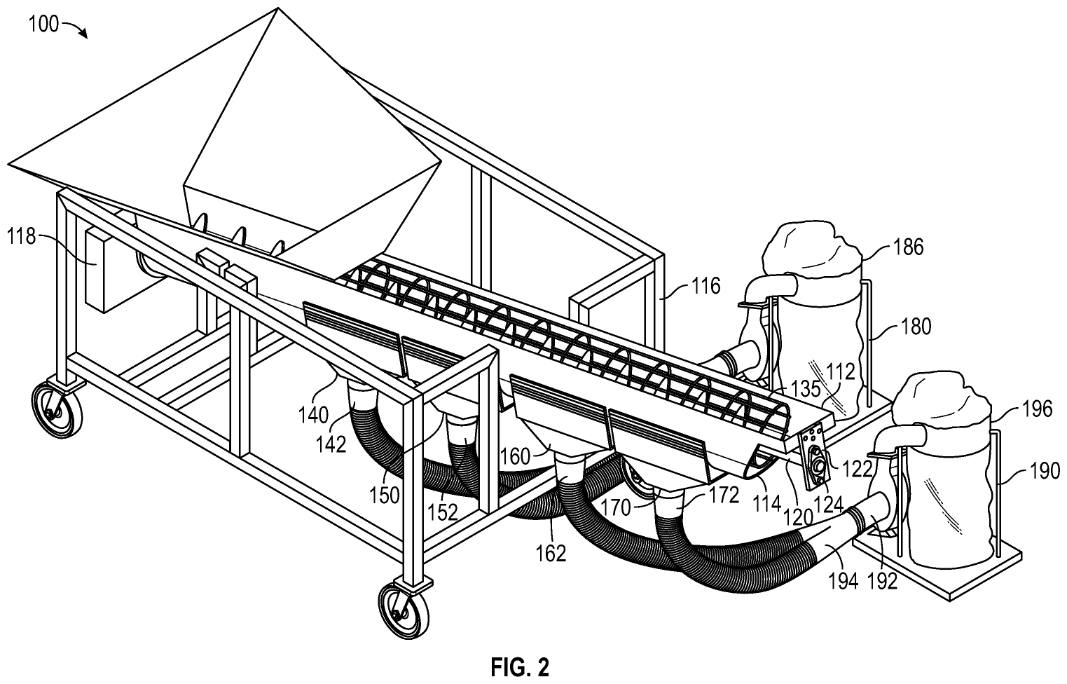

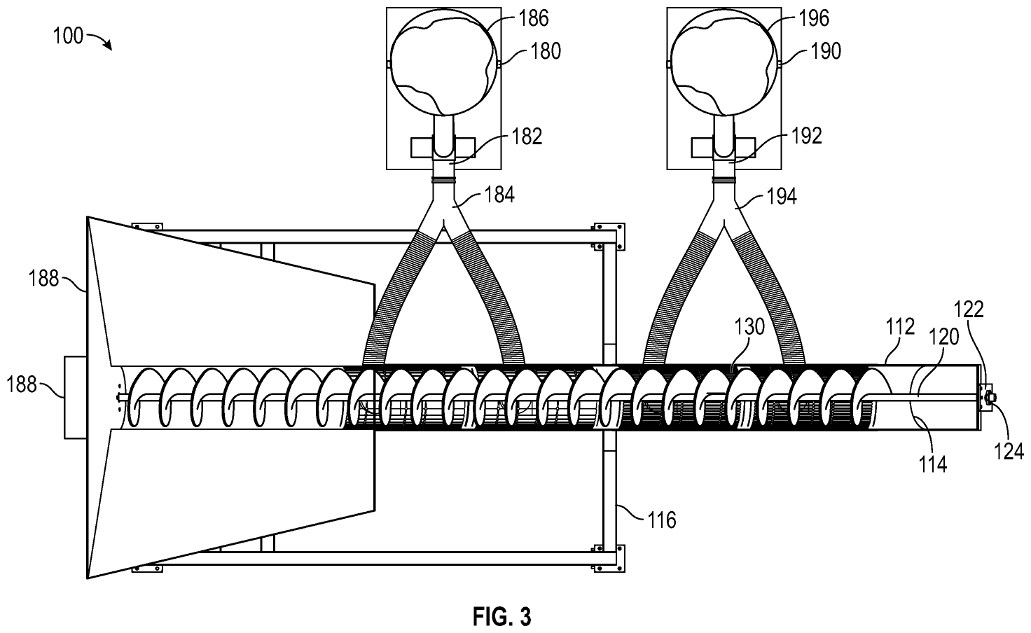

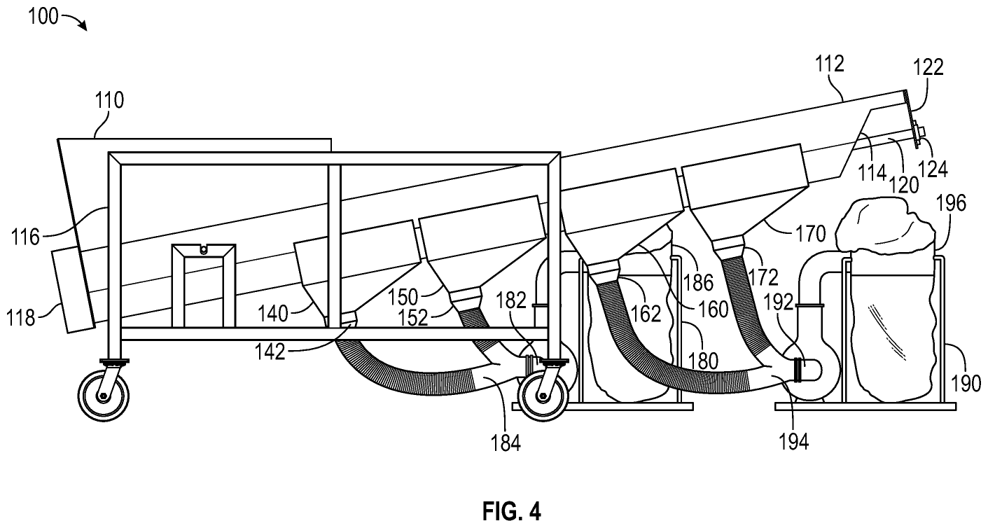

Referring to the Figures, there is shown in FIGS. 1, 2, 3, and 4 the following features:

Element 100 which is a Biomass Refinement System and Kief Extractor.

Element 110 which is a hopper.

Element 112 which is a lid for a U-shaped channel.

Element 114 which is a U-shaped channel.

Element 116 which hopper support cage.

Element 118 which is an auger drive motor device, and/or an auger and mesh drive motor device.

Element 120 which is an auger.

Element 122 which is an auger position bracket.

Element 124 which is an auger drive connector.

Element 130 which is a filter mesh.

Element 135 which is a tubular mesh.

Element 140 which is a first dust collector hood.

Element 142 which is a first dust collector vacuum connection.

Element 144 which is a first dust collector vacuum hose.

Element 150 which is a second dust collector hood.

Element 152 which is a second dust collector vacuum connection.

Element 154 which is a second dust collector vacuum hose.

Element 160 which is a third dust collector hood.

Element 162 which is a third dust collector vacuum connection.

Element 164 which is a third dust collector vacuum hose.

Element 170 which is a fourth dust collector hood.

Element 172 which is a fourth dust collector vacuum connection.

Element 174 which is a fourth dust collector vacuum hose.

Element 180 which is a first vacuum dust collector.

Element 182 which is a first vacuum.

Element 184 which is a first vacuum tube entry.

Element 186 which is a first vacuum exhaust filter.

Element 190 which is a second vacuum dust collector.

Element 192 which is a second vacuum.

Element 194 which is a second vacuum tube entry.

Element 196 which is a second vacuum exhaust filter.

In some embodiments there is a mesh, screen, or filter (not shown) covering the opening of the dust collector vacuum connection (142, 152, 162, and 172).

In all embodiments of the present invention, the deice is designed for and suitable for continuously sorting and/or processing without stopping provided that a continuous feed of raw material suitable for process by said machine is provided to the entry point of processing for the given machine.

There are at least two applications (preferred embodiments) for the present invention. The two preferred embodiments are: 1. a deleafer-separator embodiment, and 2. a biomass separator/refinement embodiment. The two embodiments comprise the same equipment/element configuration, with more filter mesh and dust collector surface area being present in the biomass-refinement embodiment.

In the first preferred embodiment there is a deleafer-separating embodiment that deleafs the female flowers and separates unwanted dried water leaves (fan leaves) from the female flowers

The deleafer-separator embodiment breaks down and separates the water leaves, also known as fan leaves from the female hemp or cannabis flowers. Note: when flowers are stripped off the branches, separating water leaves from the flowers is very labor intensive. The deleafer automatically separates the water leaves from the female flowers at a very high rate, thus saving farmers or processors from spending many many hours of manual separating. Water leaves, also called fan leaves are void of THC or CBD and therefore part of the plant not utilized.

The way the deleafer machine works is dried female flowers and water leaves are dumped into the hopper and the action of a screw auger rotating not only moves the flowers and water leaves up the u channel, but also breaks down the dried water leaves with out damaging the female cannabis flowers. As the flowers and broken down water leaves moves over the mesh at the bottom of the of the u channel where they get broken down by a grating action of moving over the rigid mesh, the water leaves then pass through and get sucked out into the dust collector. The unaffected flowers pass over the mesh and out the end thus separating the two and saving countless labor costs.

The auger action of the present invention moves flowers forward which also adds to the breaking down of the dried flowers. Also, the auger moving the flowers and dried leaves over the rigid mesh openings continues to break down water leaves and then are sucked out by vacuum dust collectors. Important to note, this method does not damage flowers as they pass through and out the end.

Biomass intended in this specification includes all the different parts of the cannabis plant. This includes, but is not limited to, the following: water leaves, sugar leaves (the tiny leaves that stick out of the female flower) leafs, small branches, stems, trichomes, crystals, and twigs. This machine will separate the sellable and usable parts of the plant from the less valuable parts. Specifically, separating the quality trim (sugar leaves) and kief (trichomes and crystals) from the stems, water leaves, and twigs. The present invention also isolates and breaks down the sugar leaves into smaller more usable size for extraction, and cannabis cigarettes. Further, the present invention isolates the kief.

The way that this is accomplished is by placing biomass into the hopper and similarly into the deleafer, the screw auger action moves and breaks down the various dried plant components and the desirable parts (quality trim and kief) gets sucked out through the mesh by and into the dust collector where the trim (sugar leaves) are broken down by the fan blades and the kief is isolated into a secondary part of the dust collector.

A difference between the deleafing embodiment/use and the biomass embodiment/use is that there is more surface area of mesh and a second set of dust collectors in the later.

Auger action moves biomass forward which also adds to the breaking down of the dried flowers. Also, the auger moving the biomass over the rigid mesh openings continues to break down water leaves which are then sucked out by vacuum and collected in the vacuum collection bags. In the biomass application the sticks, stems, water leaves, unwanted plant material passes out the end of the U-shaped channel while the good stuff (sugar leaves and kief) get sucked through mesh out into dust collectors (vacuum collection bags).

In the biomass separator/refinement embodiment the sticks, stems, water leaves, unwanted plant material passes out the end while good stuff, (sugar leaves, kief) get sucked through mesh out into dust collectors.

The apparatus of the present invention can be enhanced in speed of processing material and aggressiveness in handling of material by adding a rotating mesh cylinder around the screw auger.

In a preferred embodiment there is a deleafing device for separating dried leaves from female flowers comprising a hopper having a tray in the form a U-shaped channel having a plurality of openings positioned along a bottom with a lid covering a top side of said U-shaped channel connectively attached thereto, an auger positioned within said tray having a auger drive motor operationally attached to spin said auger about a length, a first mesh positioned around up to all of a bottom half of said length of said auger, a plurality of collection hoods positioned along a bottom side of said tray under said first mesh each having a vacuum connection, and a plurality of vacuum devices each having a collection container and a hose operationally attached to said vacuum connections.

In an alternate embodiment there is a deleafing device wherein said first mesh is position in a tube shape around said length of said auger and said first mesh further comprises a mesh drive motor operationally attached to said first mesh to spin said first mesh thereby enabling an increased speed of operation of said deleafing device.

In an alternate embodiment there is a deleafing device further comprising a second mesh having a smaller opening than said first mesh positioned under or replacing a portion of said first mesh enabling a separate vacuum device collection.

In a preferred embodiment there is a method of using a deleafing device to separate a plurality of dried leaves from a female cannabis flower of a cannabis plant comprising adding a plurality of female cannabis flowers striped off of a branch of a cannabis plant into a hopper, rotating an auger to propel said female cannabis flowers through a U-shaped channel causing said female cannabis flowers to pass over and rub against a first mesh positioned over a vacuum connection causing the separation of said dried leaves from said female cannabis flowers, and operating a plurality of vacuum devices during said rotating to collect said dried leaves.

In a preferred embodiment there is a biomass separation and refinement device for separating and refining a plurality of leaves and a plurality of kief from a biomass, consisting of all parts of a cannabis plant, comprising a hopper having a tray in the form a U-shaped channel having a plurality of openings positioned along a bottom with a lid covering a top side of said U-shaped channel connectively attached thereto, an auger positioned within said tray having a auger drive motor operationally attached to spin said auger about a length, a first mesh positioned around up to all of a bottom half of said length of said auger, a plurality of collection hoods positioned along a bottom side of said tray under said first mesh each having a vacuum connection, and a first plurality of vacuum devices and a second plurality of vacuum devices each having a collection container and a hose operationally attached to said vacuum connections.

In an alternate embodiment there is a deleafing device wherein said first mesh is position in a tube shape around said length of said auger and said first mesh further comprises a mesh drive motor operationally attached to said first mesh to spin said first mesh thereby enabling an increased speed of operation of said deleafing device.

In an alternate embodiment there is a deleafing device further comprising a second mesh having a smaller opening than said first mesh positioned under or replacing a portion of said first mesh enabling a separate vacuum device collection.

Using the deleafing application of the present invention, an advantage of the present invention is that the unwanted water leaves/fan leaves are separated from undamaged flowers which includes sugar leaves still attached to flowers.

In a preferred embodiment there is a method of using a biomass separation and refinement device to separate said plurality of leaves and said plurality of kief from a biomass comprising adding a biomass of a cannabis plant into a hopper, rotating an auger to propel said biomass through said U-shaped channel causing said biomass to pass over and rub against a first mesh positioned over a vacuum connection causing the separation of said plurality of leaves and said plurality of kief from said biomass, and operating a plurality of vacuum devices during said rotating to collect said plurality of leaves and said plurality of kief in separate vacuum devices.

In an alternate embodiment there is a method wherein said rotating an auger propels said biomass through a U-shaped channel causing said biomass to pass over and rub against a first mesh and a second mesh positioned over a vacuum connection causing the separation of said dried leaves and kief from said biomass, and operating a plurality of vacuum devices during said rotating to collect said dried leaves and said kief in separate vacuum devices.

In all embodiments of the present invention, each may be further comprising a continuous feed of a biomass for continuously separating and refining said plurality of leaves and said plurality of kief from a biomass.

In all embodiments of the present invention, each may be further comprising using said biomass separation and refinement device to separate a valuable trim from said biomass.

In all embodiments of the present invention the separate valuable trim, each may further comprising a continuous feed of a biomass for continuously separating said valuable trim from said biomass.

Using the biomass refinement application of the present invention, an advantage of the present invention is that it not only separates the sugar leaves, tiny flowers and Kief from unwanted biomass (sticks, stems, water leaves, fan leaves), it refines the sugar leaves, tiny buds, and Kief into much smaller particles.

The invention has been described by way of examples only. Therefore, the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the claims.

Although the invention has been explained in relation to various embodiments, it is to be understood that many other possible modifications and variations can be made without departing from the spirit and scope of the invention.

1. A deleafing device for separating dried leaves from female flowers comprising
a hopper having a tray in the form a U-shaped channel having a plurality of openings positioned along a bottom with a lid covering a top side of said U-shaped channel connectively attached thereto,
an auger positioned within said tray having a auger drive motor operationally attached to spin said auger about a length,
a first mesh positioned around up to all of a bottom half of said length of said auger,
a plurality of collection hoods positioned along a bottom side of said tray under said first mesh each having a vacuum connection, and
a plurality of vacuum devices each having a collection container and a hose operationally attached to said vacuum connections.

2. The deleafing device of claim 1 wherein said first mesh is position in a tube shape around said length of said auger and said first mesh further comprises a mesh drive motor operationally attached to said first mesh to spin said first mesh thereby enabling an increased speed of operation of said deleafing device.

3. The deleafing device of claim 1 further comprising
a second mesh having a smaller opening than said first mesh positioned under or replacing a portion of said first mesh enabling a separate vacuum device collection.

4. The deleafing device of claim 1 further comprising a continuous feed of a biomass for continuously separating and refining a plurality of leaves and a plurality of kief from a biomass.

5. A method of using a deleafing device to separate a plurality of dried leaves from a female cannabis flower of a cannabis plant comprising
adding a plurality of female cannabis flowers striped off of a branch of a cannabis plant into a hopper,
rotating an auger to propel said female cannabis flowers through a U-shaped channel causing said female cannabis flowers to pass over and rub against a first mesh positioned over a vacuum connection causing the separation of said dried leaves from said female cannabis flowers, and
operating a plurality of vacuum devices during said rotating to collect said dried leaves.

6. A biomass separation and refinement device for separating and refining a plurality of leaves and a plurality of kief from a biomass, consisting of all parts of a cannabis plant, comprising
a hopper having a tray in the form a U-shaped channel having a plurality of openings positioned along a bottom with a lid covering a top side of said U-shaped channel connectively attached thereto,
an auger positioned within said tray having a auger drive motor operationally attached to spin said auger about a length,
a first mesh positioned around up to all of a bottom half of said length of said auger,
a plurality of collection hoods positioned along a bottom side of said tray under said first mesh each having a vacuum connection, and
a first plurality of vacuum devices and a second plurality of vacuum devices each having a collection container and a hose operationally attached to said vacuum connections.

7. The deleafing device of claim 6 further comprising a continuous feed of a biomass for continuously separating and refining a plurality of leaves and a plurality of kief from a biomass.

8. The deleafing device of claim 6 wherein said first mesh is position in a tube shape around said length of said auger and said first mesh further comprises a mesh drive motor operationally attached to said first mesh to spin said first mesh thereby enabling an increased speed of operation of said deleafing device.

9. The deleafing device of claim 8 further comprising a continuous feed of a biomass for continuously separating and refining a plurality of leaves and a plurality of kief from a biomass.

10. The deleafing device of claim 6 further comprising
a second mesh having a smaller opening than said first mesh positioned under or replacing a portion of said first mesh enabling a separate vacuum device collection.

11. The deleafing device of claim 10 further comprising a continuous feed of a biomass for continuously separating and refining a plurality of leaves and a plurality of kief from a biomass.

12. A method of using a biomass separation and refinement device to separate said plurality of leaves and said plurality of kief from a biomass comprising
adding a biomass of a cannabis plant into a hopper,
rotating an auger to propel said biomass through said U-shaped channel causing said biomass to pass over and rub against a first mesh positioned over a vacuum connection causing the separation of said plurality of leaves and said plurality of kief from said biomass, and
operating a plurality of vacuum devices during said rotating to collect said plurality of leaves and said plurality of kief in separate vacuum devices.

13. The method of claim 12 wherein further comprising a continuous feed of a biomass for continuously separating and refining said plurality of leaves and said plurality of kief from a biomass.

14. A method as in claim 12 further comprising using said biomass separation and refinement device to separate a valuable trim from said biomass.

15. The method of claim 14 wherein further comprising a continuous feed of a biomass for continuously separating said valuable trim from said biomass.

16. The method of claim 12 wherein said rotating an auger propels said biomass through a U-shaped channel causing said biomass to pass over and rub against a first mesh and a second mesh positioned over a vacuum connection causing the separation of said dried leaves and kief from said biomass, and
operating a plurality of vacuum devices during said rotating to collect said dried leaves and said kief in separate vacuum devices.

17. The method of claim 16 wherein further comprising a continuous feed of a biomass for continuously separating and refining said plurality of leaves and said plurality of kief from a biomass.

18. A method as in claim 16 further comprising using said biomass separation and refinement device to separate a valuable trim from said biomass.

19. The method of claim 18 wherein further comprising a continuous feed of a biomass for continuously separating said valuable trim from said biomass.
